(19) United States
(12) Patent Application Publication
(10) Pub. No.: US 2016/0242660 A1
(43) Pub. Date: Aug. 25, 2016

(54) VIBRATION SENSOR AND PULSE SENSOR

(71) Applicant: KYUSHU INSTITUTE OF TECHNOLOGY, Kitakyushu-shi, Fukuoka (JP)

(72) Inventor: Yasushi SATO, Kitakyushu-shi (JP)

(73) Assignee: KYUSHU INSTITUTE OF TECHNOLOGY, Kitakyushu-shi, Fukuoka (JP)

(21) Appl. No.: 15/028,980

(22) PCT Filed: Oct. 16, 2014
(86) PCT No.: PCT/JP2014/077543

§ 371 Date: Apr. 13, 2016

(30) Foreign Application Priority Data
Oct. 18, 2013 (JP) 2013-217093

(51) Int. Cl. A61B 5/024

(52) U.S. Cl. CPC A61B 5/02444

(57) ABSTRACT

A BPF connected to a pulse wave generator has a bandwidth encompassing the fluctuation range of the resonance frequency of a helical antenna. As a result, even if the resonance frequency of the helical antenna fluctuates by a human body approaching the helical antenna, one or a number of a plurality of frequencies of signals passing through the BPF can pass through the bandwidth of the helical antenna.

### TECHNICAL FIELD

The present invention relates to a vibration sensor and a pulse sensor which utilize the Doppler effect of an electric wave.

### BACKGROUND ART

Until now, in order to detect a pulse of a human body, it has been necessary to perform sensing in a state where a sensor is brought into contact with a human body like in the case of a photoelectric pulse sensor and an electrocardiograph.

If a pulse of a human body can be detected without contact, there can be expected applications to goods for health maintenance or health care, watch sensing of a solitary senior people, and the like.

### CITATION LIST

### Patent Literature

PTL 1: Japanese Patent No. 3057438

### SUMMARY OF INVENTION

### Technical Problem

There exists a technology in which an electric wave is used as a method for detecting, without contact, an activity condition of a human body. In Patent Literature 1, a sensor of a contactless type cardiopulmonary function monitor apparatus that uses an electric wave is disclosed.

The sensor disclosed in Patent Literature 1 is one called a Doppler sensor, which is a sensor that detects the presence or the like of a target object by utilizing the Doppler effect, as has been named.

The Doppler sensor disclosed in Patent Literature 1 uses arithmetic processing by a fast Fourier transformation and a computer to thereby make an apparatus large and expensive. Accordingly, in order to apply a contactless pulse sensor to inexpensive goods, further simplification and low price are desirable.

The present invention has been made in consideration of such a situation, and an object of the present invention is to provide a vibration sensor and a pulse sensor which can detect, without contact, a low-frequency vibration of an object to be detected such as a pulse of a human body by using an extremely simple and inexpensive circuit configuration.

### Solution to Problem

In order to solve the above-described problem, an vibration sensor of the present invention includes a signal generator that generates a signal including a frequency component utilizable as an electric wave; and a band pass filter that is provided with a prescribed bandwidth and allows a signal of a frequency included in the bandwidth to pass from the signal generated by the signal generator. In addition, the vibration sensor includes a first RF amplifier that amplifies a signal obtained from the band pass filter; an antenna that emits the signal amplified by the first RF amplifier, as an electric wave; a directional coupler that is interposed between the first RF amplifier and the antenna. Furthermore, the vibration sensor includes a first mixer that multiplies a reflected wave output from the directional coupler and a progressive wave obtained from the band pass filter or the directional coupler; a second mixer that multiplies a reflected wave output from the directional coupler and a progressive wave obtained from the band pass filter or the directional coupler; and a differential amplifier that differentially amplifies an output signal of the first mixer and an output signal of the second mixer.

### Advantageous Effects of Invention

According to the present invention, a vibration sensor and a pulse sensor which can detect, without contact, a low-frequency vibration of an object to be detected such as a pulse of a human body by using an extremely simple and inexpensive circuit configuration can be provided.

Problems, configurations and effects other than those described above will become clear by the following explanations of embodiments.

### BRIEF DESCRIPTION OF DRAWINGS

FIG. 1 is a block diagram of the vibration sensor according to a first embodiment of the present invention.

FIG. 2 is a block diagram of the vibration sensor according to a second embodiment of the present invention.

FIG. 3 is an example of a circuit of a pulse wave generator.

FIG. 4 includes a wave form chart of pulse output from the pulse wave generator, a spectrum chart in the frequency region, obtained by subjecting the pulse output from the pulse wave generator to Fourier transformation, a frequency characteristic chart of a BPF, and a spectrum chart showing harmonic components passing through the BPF.

FIG. 5 includes a spectrum chart showing harmonic components passing through the BPF, and a spectrum chart showing reflected waves output from a directional coupler.

### DESCRIPTION OF EMBODIMENTS

The vibration sensor according to an embodiment of the present invention is a Doppler sensor using an electric wave. That is, a target object is irradiated with an electric wave, and the change in frequency of the reflected electric wave is detected.

However, when a target object is close to an antenna, the resonance frequency of the antenna easily fluctuates depending on the position or movement of the target object.

In the vibration sensor according to the embodiment of the present invention, electric waves of a plurality of frequencies are extracted using a band pass filter encompassing the fluctuation range of the fluctuating resonance frequency, which are utilized for detecting a low-frequency vibration.

### First Embodiment: Overall Configuration of the Vibration Sensor 101

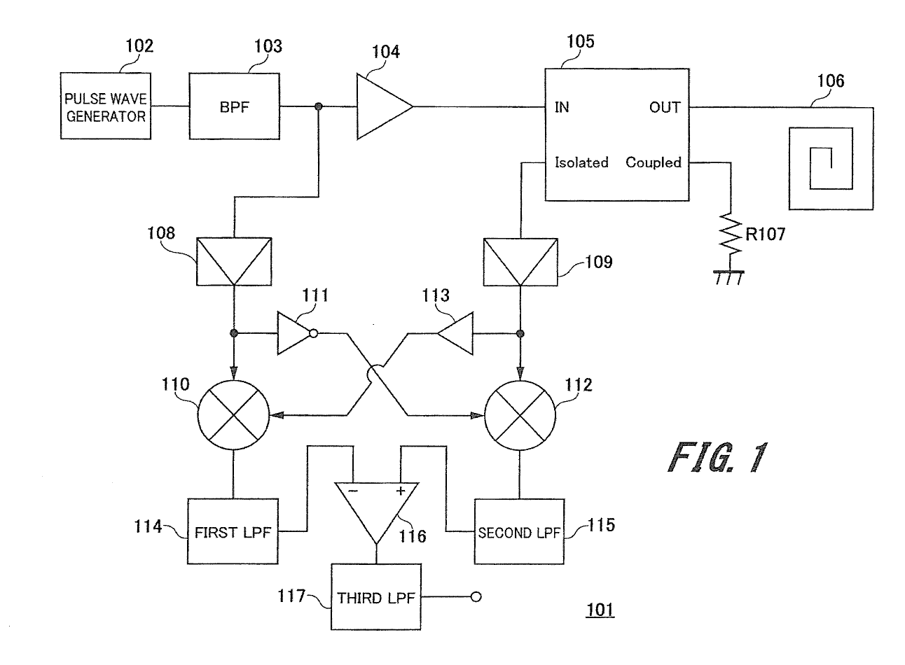

FIG. 1 is a block diagram of a vibration sensor 101 according to a first embodiment of the present invention.

The vibration sensor 101 is divided into two constituents described below.

A first constituent is a constituent that transmits an electric wave being a progressive wave to a target object, and that receives and extracts a reflected wave reflected from the target object. The first constituent includes a pulse wave generator 102, a band pass filter (hereinafter, abbreviated as “BPF”) 103, a first RF amplifier 104, a directional coupler 105 and a helical antenna 106.

A second constituent is a constituent which generates a frequency difference signal from the progressive wave and the reflected wave, and which further extracts a vibration signal. The second constituent includes a second RF amplifier 108, a third RF amplifier 109, a first mixer 110, a second mixer 112, a first low-pass filter (hereinafter, abbreviated as “LPF”) 114, a second LPF 115, a differential amplifier 116 and a third LPF 117.

The pulse wave generator 102, which may also be referred to as a signal generator, generates a pulse signal of a comparatively low frequency. The frequency to be generated by the pulse wave generator 102 is, for example, 1 MHz.

The BPF 103 takes out a harmonic component from the pulse signal generated by the pulse wave generator 102. The central frequency and bandwidth of the BPF 103 are, for example, 60 MHz±3 MHz. The BPF 103 can utilize a circuit configuration obtained by, for example, cascade-connecting LC resonance circuits.

The first RF amplifier 104 amplifies a signal of a harmonic component of a pulse signal having passed through the BPF 103.

The signal of a harmonic component of the pulse signal 1amplified by the first RF amplifier 104 is input to an input terminal of the directional coupler 105 (“IN” in FIG. 1). Then, the signal of a harmonic component of the pulse signal is supplied to the helical antenna 106 connected to an output terminal of the directional coupler 105 (“OUT” in FIG. 1).

The directional coupler 105 is formed of a coil, a condenser and a resistance, and is a well-known circuit element to be used in a VSWR meter (Voltage Standing Wave Ratio) or the like. The directional coupler 105 can output an output signal proportional to the progressive wave and an output signal proportional to the reflected wave, respectively, on the basis of a progressive wave and a reflected wave included in a first transmission path.

The helical antenna 106 emits electric waves of a plurality of frequencies based on a signal of a harmonic component of a pulse signal. Then, the electric wave reflected by a target object is received by the helical antenna 106 to thereby generate a standing wave in the inside of the directional coupler 105.

A signal that is proportional to a signal of an electric wave (reflected wave) input from the output terminal through the helical antenna 106 is output to an isolated terminal of the directional coupler 105 (“Isolated” in FIG. 1).

A signal that is proportional to the signal of a harmonic component (progressive wave) of pulse signal input to the input terminal is output to a coupled terminal of the directional coupler 105 (“Coupled” in FIG. 1).

The coupled terminal is connected to a ground node via a resistance R 107. As to a resistance value of the resistance R 107, a resistance value that is equal to an impedance of the directional coupler 105 and the helical antenna 106 is set. In many cases, the impedance of the directional coupler 105 and the helical antenna 106 is 50Ω or 75Ω.

The second RF amplifier 108 amplifies the signal of a harmonic component (progressive wave) of the pulse signal passing through the BPF 103.

The third RF amplifier 109 amplifies a signal of an electric wave (reflected wave) output from the isolated terminal of the directional coupler 105, and input from the output terminal through the helical antenna 106.

The output signal of the second RF amplifier 108 is supplied to the first mixer 110 and is supplied to the second mixer 112 via an inverting amplifier 111.

The output signal of the third RF amplifier 109 is supplied to the second mixer 112 and is supplied to the first mixer 110 via a buffer 113. Note that, even though phases may be different between the output signal of the second RF amplifier 108 and the output signal of the third RF amplifier 109, intended signals can be obtained from the first mixer 110 and the second mixer 112. Accordingly, a buffer (non-inverting amplifier) may be used instead of the inverting amplifier 111.

In this way, each of the first mixer 110 and the second mixer 112 outputs a multiplication signal of the progressive wave and the reflected wave. Here, for example, a dual gate FET or the like is utilizable as the first mixer 110 and the second mixer 112.

The output signal of the first mixer 110 is supplied to the first LPF 114. The first LPF 114 outputs a signal of difference in respective frequencies of the progressive wave and the reflected wave among the multiplication signals of the progressive wave and the reflected wave which are output from the first mixer 110.

In the same way, the output signal of the second mixer 112 is supplied to the second LPF 115. The second LPF 115 outputs a signal of difference in frequencies of the progressive wave and the reflected wave among the multiplication signals of the progressive wave and the reflected wave which are output from the second mixer 112.

Each of the output signal of the first LPF 114 and the output signal of the second LPF 115 is input to the differential amplifier 116. The differential amplifier 116 including an operational amplifier outputs an output signal of the first LPF 114 and a signal obtained by removing a noise component from an output signal of the second LPF 115.

The output signal of the differential amplifier 116 is supplied to the third LPF 117. The third LPF 117 removes an alternating-current component having a comparatively high frequency from an output signal of the differential amplifier 116 and allows a low-frequency signal exhibiting a pulse of a human body to pass through.

### Second Embodiment: Overall Configuration of Vibration Sensor 201

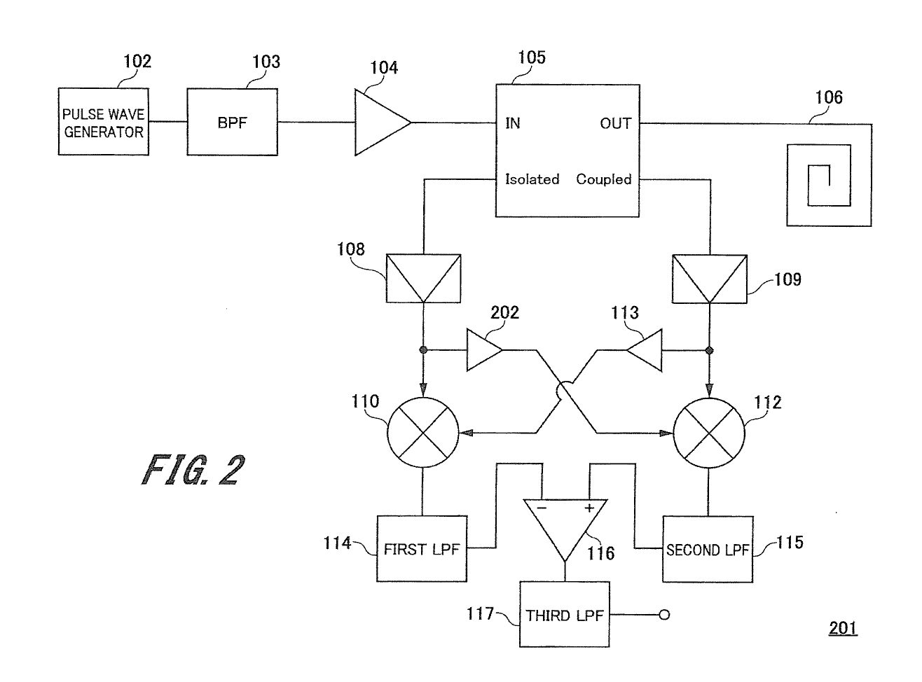

FIG. 2 is a block diagram of a vibration sensor 201 according to a second embodiment of the present invention.

The differences of the vibration sensor 201 shown in FIG. 2 from the vibration sensor 101 shown in FIG. 1 are that the input terminal of the second RF amplifier 108 is connected to the isolated terminal of the directional coupler 105, and the input terminal of the third RF amplifier 109 is connected to the coupled terminal of the directional coupler 105, respectively, and that a buffer 202 instead of the inverting amplifier 111 is connected between the second RF amplifier 108 and the second mixer 112. Note that, although the output signal of the second RF amplifier 108 and the output signal of the third RF amplifier 109 have different phases from each other, intended signals can be obtained from the first mixer 110 and the second mixer 112. Accordingly, an inverting amplifier may be used instead of the buffer 202.

That is, in the vibration sensor 201 according to the second embodiment of the present invention, the second RF amplifier 108 amplifies the reflected wave, and the third RF amplifier 109 amplifies the progressive wave.

### Specific Example of Pulse Wave Generator 102

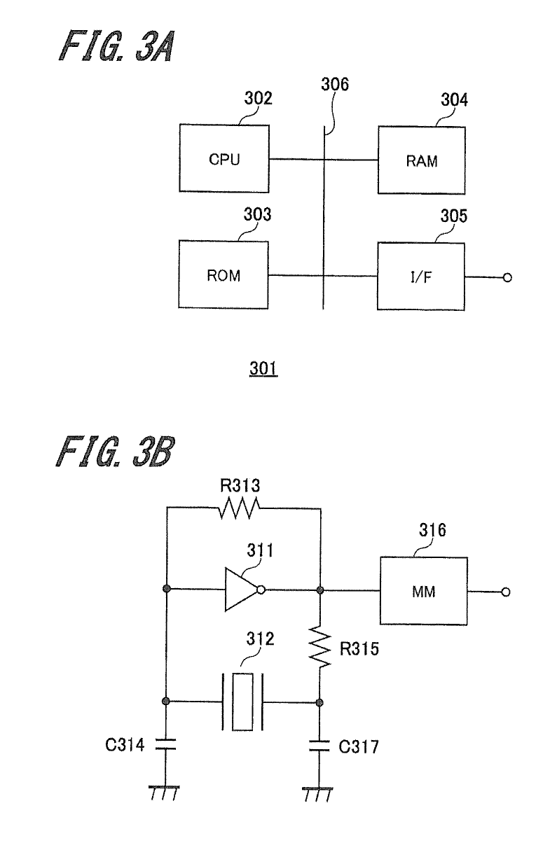

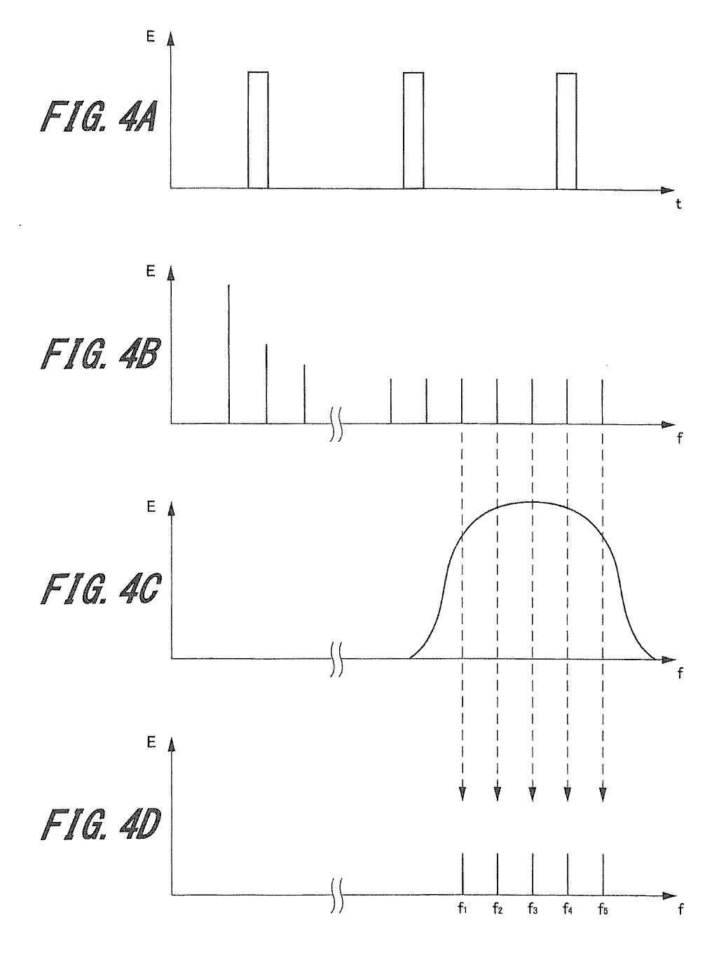

The pulse wave generator 102 that is common to the first embodiment and the second embodiment generates a pulse signal shown in FIG. 4A to be described later. Many circuits and apparatuses are considered as a method for generating such a pulse signal, and an example is shown in FIG. 3.

Each of FIG. 3A and FIG. 3B is a circuit example of the pulse wave generator 102.

FIG. 3A is a block diagram of a microcomputer 301. A CPU 302, a ROM 303, a RAM 304 and a serial interface 305 are connected to a bus 306. These days, a one-chip microcomputer that is inexpensive and excellent in usability can be easily obtained. Such one-chip microcomputer can easily generate a pulse signal as shown in FIG. 4A by writing a program in the ROM 303 being a built-in flash memory.

FIG. 3B is a circuit diagram of an oscillation circuit using a quartz oscillator and a waveform shaping circuit using a mono-multi.

One end of a quartz oscillator 312, one end of a resistance R 313 and one end of a condenser C 314 are connected to an input terminal of a NOT gate 311.

The other end of the resistance R 313 is connected to the output terminal of the NOT gate 311.

One end of a resistance R 315 and the input terminal of a mono-multi 316 are connected to the output terminal of the NOT gate 311.

The other end of the quartz oscillator 312 and one end of a condenser C 317 are connected to the other end of the resistance R 315.

Each of the other end of the condenser C 314 and the other end of the condenser C 317 is connected to the ground node.

That is, a signal generated by the oscillation circuit constituted by the NOT gate 311 and the quartz oscillator 312 is frequency-controlled by the quartz oscillator 312 and is waveform-shaped into a pulse signal whose duty ratio has been adjusted by the mono-multi 316.

[Operation of Vibration Sensor 101]

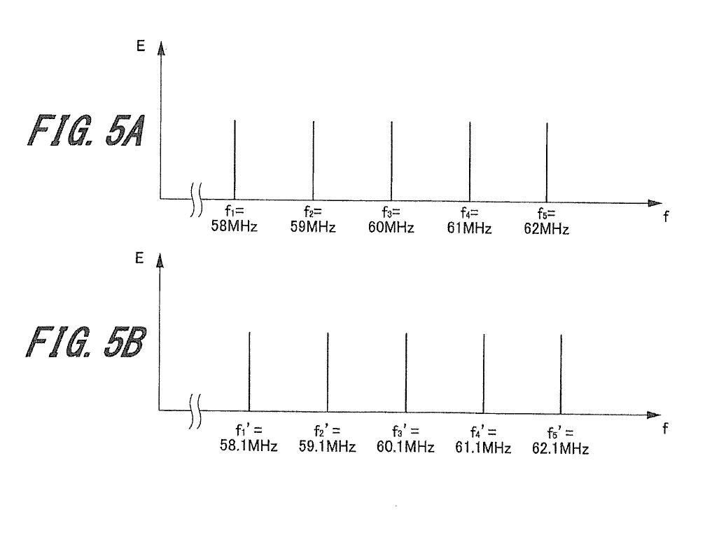

Hereinafter, operations of the vibration sensor 101 will be explained with reference to FIG. 4A, FIG. 4B, FIG. 4C, FIG. 4D, FIG. 5A and FIG. 5B.

FIG. 4A is a wave form chart of a pulse signal output from the pulse wave generator 102. The horizontal axis of the wave form chart represents time and the vertical axis represents a voltage. As shown in FIG. 4A, a waveform that has a small duty ratio and is close to an impulse contains a lot of harmonics, and thus such a waveform is desirable for the vibration sensor 101 of the embodiment.

FIG. 4B is a spectrum chart in the frequency region obtained by subjecting the pulse signal shown in FIG. 4A output from the pulse wave generator 102, to Fourier transformation. The horizontal axis of the spectrum chart represents a frequency, and the vertical axis represents a voltage. As shown in FIG. 4B, the pulse signal includes a plurality of harmonics having frequencies of integral multiples relative to the fundamental wave.

FIG. 4C is a frequency characteristic chart of the BPF 103. The scale in FIG. 4C is adjusted to that in FIG. 4B, and thus the horizontal axis of the frequency characteristic chart represents a frequency and the vertical axis represents a voltage.

FIG. 4D is a frequency distribution chart of signals having passed through the BPF 103. The scale in FIG. 4D is also adjusted to that in FIG. 4C, the horizontal axis of the frequency characteristic chart represents a frequency and the vertical axis represents a voltage.

As shown in FIG. 4C, the BPF 103 allows a component of a specific frequency to pass through among harmonic components included in the pulse signal. Then, as shown in the frequency distribution chart in FIG. 4D, in the harmonic component of the pulse signal having passed through the BPF 103, frequency components or the like having a cutoff frequency or less including the fundamental wave are removed from the pulse signal.

FIG. 5A shows enlarging the frequency axis (horizontal axis) of the frequency distribution chart in FIG. 4D, which is a spectrum chart representing harmonic components of the pulse signal having passed through the BPF 103.

FIG. 5B is a spectrum chart showing the reflected wave output from the directional coupler 105.

Now, as shown in FIG. 5A, harmonic components of the pulse signal having passed through the BPF 103 are assumed to be five signals centering on 60 MHz. Five signals are f1=58 MHz, f2=59 MHz, f3=60 MHz, f4=61 MHz and f5=62 MHz in ascending order of frequencies. These five signals are amplified by the first RF amplifier 104, and the amplified signals are emitted as electric waves from the helical antenna 106 via the directional coupler 105.

However, since the frequency characteristic (bandwidth) of the helical antenna 106 is narrow, any one or approximately two of signals of f1 to f5 are emitted as electric waves from the helical antenna 106.

Then, electric waves emitted from the helical antenna 106 are reflected by a target object and input to the directional coupler 105 through the helical antenna 106. Signals of these reflected waves are, for example as shown in FIG. 5B, any of f1′=58.1 MHz, f2′=59.1 MHz, f3′=60.1 MHz, f4′=61.1 MHz and f5′=62.1 MHz in ascending order of frequencies. In the example, it is assumed that the frequency of the reflected wave has shifted by 100 kHz from the progressive wave by the Doppler effect.

Any of f1 to f5 and any of f1′ to f5′ are input to the first mixer 110 and the second mixer 112, and are subjected to multiplication. Then, the first mixer 110 and the second mixer 112 output signals obtained by adding respective frequencies and signals obtained by subtracting respective frequencies.

Signals obtained by adding a frequency include, for example, f1+f1′, f2+f1′, . . . and f5+f1′ when a reflected wave includes f1′.

When the reflected wave is f2′, the signals include f1+f2′, f2+f2′ . . . and f5+f2′.

In the same way, cases when a reflected wave includes f3′, . . . and a reflected wave includes f4′ follow the above, and when a reflected wave includes f5′, the signals include f1+f5′, f2+f5′ . . . and f5+f5′.

Signals obtained by subtracting a frequency include, for example, |f1−f1′|, |f2−f1′| . . . and |f5−f1′| when a reflected wave includes f1′.

When a reflected wave includes f2′, the signals include |f1−f2′|, |f2−f2′| . . . and |f5−f2′|.

In the same way, cases when a reflected wave includes f3′, . . . and a reflected wave includes f4′ follow the above, and when a reflected wave includes f5′, the signals include |f1−f5′|, |f2−f5′| . . . and |f5−f5′|.

The lowest frequency among signals output from the first mixer 110 and the second mixer 112 is |f1−f1′|, |f2−f2′|, |f3−f3′|, |f4−f4′| and |f5−f5′|. In the cases of FIG. 5A and FIG. 5B, all frequencies of these signals are 100 kHz. These signals have only a component resulting from the shift of frequency by the Doppler effect, and all frequencies become identical.

When a target object is close to an antenna, the resonance frequency of the antenna easily fluctuates depending on the position or action of the target object. Consequently, even when an electric wave is emitted from the antenna by using a signal of a single frequency, the signal generates mismatch with the resonance frequency of the antenna, and a reflected wave cannot be correctly received.

Therefore, the vibration sensor according to the embodiment of the present invention utilizes electric waves of a plurality of frequencies using a band pass filter encompassing the fluctuation of the resonance frequency. As a result, even when the resonance frequency of the antenna fluctuates, any one or approximately two of signals of a plurality of frequencies coincide with the bandwidth of the antenna and the reflected wave can be received.

When the reflected wave can be received, the presence and/or fluctuation state of the target object can be detected by taking out, by using a mixer, the frequency difference between the reflected wave and the progressive wave generated by the Doppler effect.

Note that 60 MHz is a frequency that allegedly matches most easily with the blood flow of a human body.

Both the vibration sensor 101 according to the first embodiment of the present invention and the vibration sensor 201 according to the second embodiment remove noise of an in-phase component included in a signal by using the differential amplifier 116. Furthermore, these sensors also remove noise of a high-frequency component by causing the signal to pass through the third LPF 117. As a result of removing these noises, the vibration sensor 101 and the vibration sensor 201 of the embodiment can detect vibration from a weak fluctuation generated on electric waves by vibration of a target object, without using an expensive apparatus such as high-speed Fourier transformation.

Application examples as described below are possible in embodiments having been described above.

(1) In the above-described embodiments, the helical antenna 106 has been used, but the type of antennas is not limited to this. There can be used any antenna having an open end, such as a dipole antenna, a ground plane antenna, and a meander line antenna. Furthermore, even a loop antenna not having an open end is utilizable although gain lowers.

(2) In place of the pulse wave generator 102, a circuit that generates white noise may be used.

(3) The vibration sensors 101 and 201 of the embodiments detect, without contact, a low-frequency vibration of an object. An object to be detected is not limited to a specific object as long as it is an object that can cause a change in the distribution constant of an antenna by approaching the antenna. Accordingly, the vibration sensors 101 and 201 can also be used as a pulse sensor.

(4) The vibration sensors 101 and 201 according to the embodiments can be applied to various applications. For example, there can be expected an application as a driver dozing preventing apparatus that detects a driver's doze by locating a pulse sensor on a driver's seat of a car. In addition, variations can also be given to game development by locating a pulse sensor on a game machine and detecting a pulse of a player to thereby give analogy of the excitation degree. Furthermore, it is also possible to realize a pulse detection device that detects a pulse, when providing a desk and a chair at a corner of a room by using a pulse sensor together with a human detection sensor, and immediately when detecting that a human has sat on the chair. The pulse detection device can detect a pulse more simply and in a shorter time than a conventional blood pressure gauge.

In the above-described embodiments, there has been explained the vibration sensor 101 in which an electric wave (progressive wave) is emitted from an antenna by using signals of a plurality of frequencies, an electric wave (reflected wave) reflected from a human body being a target object is taken out using the directional coupler 105, and a frequency difference signal between the progressive wave and the reflected wave is extracted using a mixer.

The BPF 103 connected to the pulse wave generator 102 has a bandwidth encompassing the fluctuation range of the resonance frequency of the helical antenna 106. Accordingly, even if the resonance frequency of the helical antenna 106 fluctuates by means of a target object approaching the helical antenna 106, one or a number of a plurality of frequencies passing through the BPF 103 can pass through the bandwidth of the helical antenna 106. Consequently, the vibration sensor being a Doppler sensor using an electric wave of a low frequency can be realized.

In the vibration sensors 101 and 201 of the embodiments, frequencies of signals to be handled are as low as approximately several dozen to several hundred MHz, as compared with conventional Doppler sensors. Consequently, the price of the circuit element is inexpensive. Furthermore, since frequencies are low, the implementation of circuits including the BPF 103 and the directional coupler 105 is easy. In addition, since frequencies of signals to be handled are low, only a small amount of electric power is consumed.

Moreover, the vibration sensor 101 of the embodiment has an extremely small circuit scale, as compared with conventional Doppler sensors.

One transistor is required for each of the first RF amplifier 104, the second RF amplifier 108 and the third RF amplifier 109, and the buffer 113 and the inverting amplifier 111.

One dual gate FET is also required for each of the first mixer 110 and the second mixer 112.

One inexpensive one-chip microcomputer is required for the pulse wave generator 102.

The vibration sensors 101 and 201 of the embodiments do not require Fourier transformation and complicated data processing, differently from the technology disclosed in Patent Literature 1.

In this way, the vibration sensors 101 and 201 of the embodiments can be implemented with semiconductor elements being active elements having a number less than ten in total. Accordingly, they can be manufactured inexpensively and be easily mass-produced.

Hereinbefore, although embodiments of the present invention have been explained, the present invention is not limited to the above-described embodiments, and includes other modifications and application examples as long as they do not deviate from the gist of the present invention described in the claims.

For example, the above-described embodiments are those in which the configuration of apparatuses and systems are explained in detail and in concrete terms for simply explaining the present invention, and embodiments are not necessarily limited to those provided with all configurations having been explained. Additionally, it is possible to substitute a part of the configuration of an embodiment with the configuration of another embodiment, and furthermore, it is also possible to add the configuration of another embodiment to the configuration of an embodiment. Moreover, it is also possible to perform addition of another configuration, elimination or substitution, for a part of the configurations of the respective embodiments.

In addition, a part or the whole of the above-described respective configurations, functions, processing units or the like may be realized with hardware, for example, by designing these through the use of an integrated circuit. Furthermore, the above-described respective configurations, functions or the like may be realized with software for interpreting and executing a program that causes a processer to realize respective functions. Information such as a program for realizing the respective functions, tables and files can be stored on a volatile or nonvolatile storage such as a memory, a hard disk, or an SSD (Solid State Drive); or on a recording medium such as an IC card or optical disk.

Moreover, control lines and information lines indicate lines that are considered to be necessary for explanation, and do not necessarily indicate all control lines and information lines in products. Actually almost all configurations may be considered to be mutually connected.

### REFERENCE SIGNS LIST

101: vibration sensor, 102: pulse wave generator, 103: BPF, 104: first RF amplifier, 105: directional coupler, 106: helical antenna, 108: second RF amplifier, 109: third RF amplifier, 110: first mixer, 111: inverting amplifier, 112: second mixer, 113: buffer, 114: first LPF, 115: second LPF, 116: differential amplifier, 117: third LPF, 201: vibration sensor, 202: buffer, 301: microcomputer, 312: quartz oscillator, 316: mono-multi

1. A vibration sensor comprising:
a signal generator that generates a signal including a frequency component utilizable as an electric wave;
a band pass filter that is provided with a prescribed bandwidth and allows a signal of a frequency included in the bandwidth to pass from the signal generated by the signal generator;
a first RF amplifier that amplifies a signal obtained from the band pass filter;
an antenna that emits the signal amplified by the first RF amplifier, as an electric wave;
a directional coupler that is interposed between the first RF amplifier and the antenna;
a first mixer that multiplies a reflected wave output from the directional coupler and a progressive wave obtained from the band pass filter or the directional coupler;
a second mixer that multiplies a reflected wave output from the directional coupler and a progressive wave obtained from the band pass filter or the directional coupler; and
a differential amplifier that differentially amplifies an output signal of the first mixer and an output signal of the second mixer.

2. The vibration sensor according to claim 1, wherein a bandwidth of the band pass filter encompasses a fluctuation range of a resonance frequency of the antenna.

3. The vibration sensor according to claim 1, further comprising:
a second RF amplifier that amplifies the reflected wave or the progressive wave;
a third RF amplifier that forms a pair with the second RF amplifier and amplifies the progressive wave or the reflected wave;
a first low-pass filter that removes a signal of an unnecessary high-frequency component from an output signal of the first mixer;
a second low-pass filter that removes a signal of an unnecessary high-frequency component from an output signal of the second mixer; and
a third low-pass filter that removes a signal of an unnecessary high-frequency component from an output signal of the differential amplifier.

4. The vibration sensor according to claim 3, wherein:
the second RF amplifier amplifies an output signal of the band pass filter; and
the third RF amplifier amplifies an output signal of the directional coupler.

5. The vibration sensor according to claim 3, wherein:
the second RF amplifier amplifies an output signal that is output from an isolated terminal of the directional coupler; and
the third RF amplifier amplifies an output signal that is output from a coupled terminal of the directional coupler.

6. The vibration sensor according to claim 1 wherein the signal generator generates a pulse wave.

7. A pulse sensor that detects a pulse of a human body by using a vibration sensor according to claim 1.

8. A pulse sensor that detects a pulse of a human body by using a vibration sensor according to claim 2.

9. A pulse sensor that detects a pulse of a human body by using a vibration sensor according to claim 3.

10. A pulse sensor that detects a pulse of a human body by using a vibration sensor according to claim 4.

11. A pulse sensor that detects a pulse of a human body by using a vibration sensor according to claim 5.

12. A pulse sensor that detects a pulse of a human body by using a vibration sensor according to claim 6.
